(19) United States
(12) Patent Application Publication
(10) Pub. No.: US 2026/0000458 A1
(43) Pub. Date: Jan. 1, 2026

(54) PREDICTING REQUIREMENT FOR A PACEMAKER POST TRANSCATHETER AORTIC VALVE REPLACEMENT

(71) Applicant: Libra Science Ltd., Road Town (VG)

(72) Inventor: Shlomo BEN-HAIM, Milan (IT)

(73) Assignee: Libra Science Ltd., Road Town (VG)

(21) Appl. No.: 19/250,149

(22) Filed: Jun. 26, 2025

Related U.S. Application Data
(60) Provisional application No. 63/664,786, filed on Jun. 27, 2024.

(51) Int. Cl. A61B 34/10; G06T 7/11; G06T 7/60; G16H 10/60; G16H 20/40; G16H 50/70

(52) U.S. Cl. CPC A61B 34/10; G06T 7/11; G06T 7/60; G16H 10/60; G16H 20/40; G16H 50/70; A61B 2034/104; A61B 2034/105; A61B 2034/107; G06T 2207/30048

(57) ABSTRACT

There is provided a computer implemented method of predicting likelihood of a subject requiring a pacemaker after a transcatheter aortic valve replacement (TAVR) procedure, comprising: computing from a 3D image of a subject at least one parameter selected from: (i) a depth of a membranous septum computed as a distance between the membranous septum and a virtual annulus plane of a native aortic valve, (ii) an angle of rotation of at least one cusp of the native aortic valve relative to the membranous septum, and (iii) a left ventricle (LV)—aorta angulation, and computing a prediction of likelihood of the subject requiring the pacemaker after the TAVR according to the at least one parameter.

### RELATED APPLICATIONS

This application claims the benefit of priority of U.S. Provisional Patent Application No. 63/664,786 filed on Jun. 27, 2024, the contents of which are all incorporated by reference as if fully set forth herein in their entirety.

This application is also related to PCT International Patent Application Nos. PCT/IB2025/050367 filed on Jan. 13, 2025, titled “PERSONALIZED SAFE ZONE FOR A TAVI PROCEDURE”; PCT/IB2024/057540 filed on Aug. 4, 2024, titled “CARDIAC CONDUCTION SYSTEM IMAGING” and PCT/IB2024/057539 filed on Aug. 4, 2024, titled “SYSTEM AND METHODS FOR SUPPORTING A TAVI PROCEDURE”, the contents of which are all incorporated by reference as if fully set forth herein in their entirety.

### BACKGROUND

The present invention, in some embodiments thereof, relates to Transcatheter Aortic Valve Replacement (TAVR) and, more specifically, but not exclusively, to predicting whether a subject will require a pacemaker after TAVR.

A TAVR procedure involves insertion of a catheter into the heart and expansion of an aortic valve prosthesis device within the heart to replace the native aortic valve. During manipulation of the catheter and/or the aortic valve prosthesis device within the heart, the conduction system may become damaged, requiring implantation of a pacemaker to correct conduction disturbances arising from the damage.

### SUMMARY

According to some embodiments of the present invention, there is provided a computer implemented method of predicting likelihood of a subject requiring a pacemaker after a transcatheter aortic valve replacement (TAVR) procedure, comprising: computing from a 3D image of a subject at least one parameter selected from: (i) a depth of a membranous septum computed as a distance between the membranous septum and a virtual annulus plane of a native aortic valve, (ii) an angle of rotation of at least one cusp of the native aortic valve relative to the membranous septum, and (iii) a left ventricle (LV)-aorta angulation, and computing a prediction of likelihood of the subject requiring the pacemaker after the TAVR according to the at least one parameter.

Optionally, wherein the angle of rotation is computed from a reference point located in the middle of the native aortic valve, by computing a first line from a point on the native aortic valve to the reference point, a second line from a location of the membranous septum to the reference point, and wherein the angle of rotation is an angle between the first line and the second line.

Optionally, the location of the membranous septum is a middle of the membranous septum.

Optionally, the point on the native aortic valve is at a commissure between a right coronary cusp (RCC) and a non-coronary cusp (NCC).

Optionally, the distance between a perforating bundle and a branching bundle overlying the membranous septum floor is used as an indicator for the location of a conduction pathway overlying the membranous septum floor, wherein the depth of the membranous septum floor is to the location of the conduction pathway.

Optionally, the at least one parameter further includes a depth of a conduction pathway computed as a distance between the conduction pathway overlying the membranous septum floor and a virtual annulus plane of a native aortic valve, wherein the conduction pathway is between a perforating bundle and a branching bundle.

Optionally, the prediction of likelihood is computed according to a value of the depth of the conduction pathway, selected from: (i) a negative depth of the conduction pathway below zero indicating a high risk for implantation of pacemaker post TAVR, (ii) a high positive depth of the conduction pathway greater than a threshold indicating a lower risk for implantation of pacemaker post TAVR, and (iii) a low positive depth of the conduction pathway between zero and the threshold, indicating an intermediate risk for implantation of pacemaker post TAVR.

Optionally, the at least one parameter further includes an indication of whether the subject is experiencing a right bundle branch block.

Optionally, further comprising generating a 3D model based on the 3D image of the subject and according to the at least one parameter, the 3D model simulating physical forces applied according to the at least one parameter on a virtual elongated tool for delivery of an aortic valve prosthesis device for implant during the TAVR procedure, further comprising analyzing the 3D model to compute a distance from the virtual elongated tool to the membranous septum, wherein the prediction is based on the distance.

Optionally, the physical forces are applied to the virtual elongated tool by curvature of an ascending aorta, and according to the anatomy of the heart defined by the at least one parameter, for curving the virtual elongated tool and pushing the virtual elongated tool into a location within the native aortic valve.

Optionally, a path of the virtual elongated tool inserted into a left ventricle from the aorta, is computed using the at least one parameter.

Optionally, the path of the virtual elongated tool inserted into the left ventricle from the aorta is computed, is used to predict the interaction of the tool with the membranous septum.

Optionally, computing the prediction comprises feeding the at least one parameter into a machine learning model, and obtaining the prediction from the machine learning model.

Optionally, the machine learning model is trained on a plurality of records created for a plurality of sample individuals, wherein a record for a sample individual created is created by: computing the at least one parameter from a sample 3D image of the sample individual, accessing an indication of whether the subject required a pacemaker after the TAVR, and creating the record including the at least one parameter computed for the sample individual, and a ground truth label of the indication of whether the subject required the pacemaker after the TAVR.

Optionally, further comprising: in response to computing the prediction that the subject will need the pacemaker after the TAVR, generating a message including a recommendation to implant the pacemaker prior to the TAVR and/or implanting the pacemaker prior to the TAVR.

Optionally, the prediction is computed by a heuristic process that uses two or three of the at least one parameter.

Optionally, further comprising: analyzing the 3D image for identifying a first location of a perforating bundle of a conduction system of the heart and a second location of a branching point of a left bundle branch (LBB) of the conduction system, wherein the depth of the membranous septum is computed as a depth to a cardiac conduction region defined between the first location and the second location.

Optionally, further comprising segmenting the membranous septum, wherein the depth is computed for at least one of: a most anterior point of the membranous septum, a most posterior point of the membranous septum, a center of a line between the most anterior and most posterior points, and a center of mass of the segmented membranous septum.

Optionally, further comprising: computing the virtual annulus plane with respect to the 3D image, segmenting the membranous septum on the 3D image, and computing the depth as a distance from the virtual annulus plane to the segmented membranous septum.

Optionally, computing the virtual annulus comprises: identifying three nadirs of three cusps of the native aortic valve within the 3D image, and computing the virtual annulus as a plane intersecting the three nadirs.

Optionally, the rotation angle is computed along a circumference of the aorta from a point along the native aortic valve to the membranous septum.

Optionally, further comprising: performing a multi-series 3D imaging session, wherein a plurality of 3D images depicting at least the native aortic valve are captured at different phases of a cardiac cycle, detecting a location of at least one of the following in the plurality of 3D images depicting the different phases of the cardiac cycle: the membranous septum, commissures of the native aortic valve, nadirs of the native aortic valve, and a cardiac conduction region defined between a perforating bundle of a conduction system of the heart and a branching point of a left bundle branch (LBB) of the conduction system, computing the depth of the membranous septum and/or the angle of rotation for each of the plurality of 3D images depicting the different phases of the cardiac cycle, and computing a minimum value of the depth of the membranous and/or minimum value for the angle of rotation according to the depth and/or angle computed using the plurality of 3D images, wherein the depth of the membranous septum comprises the minimum value of the depth, and the angle of rotation comprises the minimum value of the angle.

Optionally, further comprising: segmenting a right ventricle endocardium (RV), a RV myocardium, a left ventricle (LV) endocardium, and a LV myocardium, identifying a region between the RV myocardium and the LV myocardium includes a minimum spatially consistent distance between the RV myocardium and the LV myocardium, wherein the depth of the membranous septum comprises the depth of the region.

Optionally, further comprising: detecting a posterior down slope of a notch on the LV myocardium below an aortic root, and verifying that the region corresponds to the posterior down slope of the notch of the LV myocardium.

Optionally, further comprising: detecting cardiac conduction region defined between a perforating bundle of a conduction system of the heart and a branching point of a left bundle branch (LBB) of the conduction system with the region, wherein the depth of the membranous septum comprises a depth of the cardiac conduction region.

Optionally, computing the prediction comprises classifying the at least one parameter into a classification category selected from high likelihood of damage to the cardiac conduction system indicating a recommendation for pre-operative implantation of the pacemaker, and low likelihood of damage to the cardiac conduction system that does not justify the pre-operative implantation of the pacemaker.

According to some embodiments of the present invention, there is provided a computer implemented method of training a machine learning model for predicting likelihood of a subject requiring a pacemaker after a TAVR procedure, comprising: creating a training dataset of a plurality of records of a plurality of individuals, wherein a record of an individual includes: at least one parameter computed from a sample 3D image of the individual, the at least one parameter selected from: (i) a depth of a membranous septum computed as a distance between the membranous septum and a virtual annulus plane of a native aortic valve, (ii) an angle of rotation of at least one cusp of the native aortic valve relative to the membranous septum, (iii) a left ventricle (LV)-aorta angulation, (iv) a depth of a conduction pathway computed as a distance between the conduction pathway overlying the membranous septum floor and a virtual annulus plane of a native aortic valve, wherein the conduction pathway is between a perforating bundle and a branching bundle, and a ground truth indicating whether the individual required implantation of a pacemaker after the TAVR or did not require the pacemaker, and training a machine learning model on the training dataset.

According to some embodiments of the present invention, there is provided a computer implemented method of predicting likelihood of a subject requiring a pacemaker after a transcatheter aortic valve replacement (TAVR) procedure, comprising: accessing a 3D image of a subject depicting at least a native aortic valve of the subject and anatomical structures in proximity to the native aortic valve, generating a 3D model based on the 3D image of the subject, simulating physical forces applied by the 3D model on a virtual elongated tool for delivery of an aortic valve prosthesis device for implant during the TAVR procedure, computing a distance from the virtual elongated tool to a membranous septum detected on the 3D model, and computing a prediction of whether the subject will need a pacemaker after the TAVR according to the distance.

Optionally, the physical forces are applied to the virtual elongated tool by curvature of an ascending aorta, by the structure of the native aortic valve, and according to a rotation angle between at least one cusp of the native aortic valve and a membranous septum, for curving the virtual elongated tool and pushing the virtual elongated tool into a location within the native aortic valve.

Optionally, the subject is predicted to need the pacemaker when the distance is below a threshold.

According to some embodiments of the present invention, there is provided a method of treating a patient undergoing a TAVR procedure, comprising: computing from a 3D image of a subject depicting at least a native aortic valve of the subject and anatomical structures in proximity to the native aortic valve, at least one parameter selected from: (i) a depth of a membranous septum computed as a distance between the membranous septum and a virtual annulus plane of a native aortic valve, (ii) an angle of rotation of at least one cusp of the native aortic valve relative to the membranous septum, (iii) a left ventricle (LV)-aorta angulation, (iv) a depth of a conduction pathway computed as a distance between the conduction pathway overlying the membranous septum floor and a virtual annulus plane of a native aortic valve, wherein the conduction pathway is between a perforating bundle and a branching bundle, computing a prediction of likelihood of the subject requiring a pacemaker after the TAVR according to the at least one parameter, and in response to the prediction that the subject will need the pacemaker after the TAVR, treating the patient by implanting the pacemaker prior to the TAVR.

According to some embodiments of the present invention, there is provided a system for predicting likelihood of a subject requiring a pacemaker after a transcatheter aortic valve replacement (TAVR) procedure, comprising: at least one processor executing a code for: computing from a 3D image of a subject at least one parameter selected from: (i) a depth of a membranous septum computed as a distance between the membranous septum and a virtual annulus plane of a native aortic valve, (ii) an angle of rotation of at least one cusp of the native aortic valve relative to the membranous septum, and (iii) a left ventricle (LV)-aorta angulation, and computing a prediction of likelihood of the subject requiring the pacemaker after the TAVR according to the at least one parameter.

Optionally, the at least one parameter further includes a depth of a conduction pathway computed as a distance between the conduction pathway overlying the membranous septum floor and a virtual annulus plane of a native aortic valve, wherein the conduction pathway is between a perforating bundle and a branching bundle.

Optionally, the prediction of likelihood is computed according to a value of the depth of the conduction pathway, selected from: (i) a negative depth of the conduction pathway below zero indicating a high risk for implantation of pacemaker post TAVR, (ii) a high positive depth of the conduction pathway greater than a threshold indicating a lower risk for implantation of pacemaker post TAVR, and (iii) a low positive depth of the conduction pathway between zero and the threshold, indicating an intermediate risk for implantation of pacemaker post TAVR.

According to some embodiments of the present invention, there is provided a system for predicting likelihood of a subject requiring a pacemaker after a transcatheter aortic valve replacement (TAVR) procedure, comprising: at least one processor executing a code for: receiving at least one parameter of the subject selected from: (i) a depth of a membranous septum computed as a distance between the membranous septum and a virtual annulus plane of a native aortic valve, (ii) an angle of rotation of at least one cusp of the native aortic valve relative to the membranous septum, and (iii) a left ventricle (LV)-aorta angulation, and computing a prediction of likelihood of the subject requiring the pacemaker after the TAVR according to the at least one parameter.

Optionally, the angle of rotation is computed from a reference point located in the middle of the native aortic valve, by computing a first line from a point on the native aortic valve to the reference point, a second line from a location of the membranous septum to the reference point, and wherein the angle of rotation is an angle between the first line and the second line.

Optionally, the location of the membranous septum is a middle of the membranous septum.

Optionally, the point on the native aortic valve is at a commissure between a right coronary cusp (RCC) and a non-coronary cusp (NCC).

Optionally, the distance between a perforating bundle and a branching bundle overlying the membranous septum floor is used as an indicator for the location of a conduction pathway overlying the membranous septum floor, wherein the depth of the membranous septum floor is to the location of the conduction pathway.

Optionally, the at least one parameter further includes a depth of a conduction pathway computed as a distance between the conduction pathway overlying the membranous septum floor and a virtual annulus plane of a native aortic valve, wherein the conduction pathway is between a perforating bundle and a branching bundle.

Optionally, the prediction of likelihood is computed according to a value of the depth of the conduction pathway, selected from: (i) a negative depth of the conduction pathway below zero indicating a high risk for implantation of pacemaker post TAVR, (ii) a high positive depth of the conduction pathway greater than a threshold indicating a lower risk for implantation of pacemaker post TAVR, and (iii) a low positive depth of the conduction pathway between zero and the threshold, indicating an intermediate risk for implantation of pacemaker post TAVR.

Optionally, the at least one parameter further includes an indication of whether the subject is experiencing a right bundle branch block.

Optionally, further comprising code for generating a 3D model based on the 3D image of the subject and according to the at least one parameter, the 3D model simulating physical forces applied according to the at least one parameter on a virtual elongated tool for delivery of an aortic valve prosthesis device for implant during the TAVR procedure, further comprising analyzing the 3D model to compute a distance from the virtual elongated tool to the membranous septum, wherein the prediction is based on the distance.

Optionally, the physical forces are applied to the virtual elongated tool by curvature of an ascending aorta, and according to the anatomy of the heart defined by the at least one parameter, for curving the virtual elongated tool and pushing the virtual elongated tool into a location within the native aortic valve.

Optionally, a path of the virtual elongated tool inserted into a left ventricle from the aorta, is computed using the at least one parameter.

Optionally, the path of the virtual elongated tool inserted into the left ventricle from the aorta is computed, is used to predict the interaction of the tool with the membranous septum.

Optionally, computing the prediction comprises feeding the at least one parameter into a machine learning model, and obtaining the prediction from the machine learning model.

Optionally, the machine learning model is trained on a plurality of records created for a plurality of sample individuals, wherein a record for a sample individual created is created by: computing the at least one parameter from a sample 3D image of the sample individual, accessing an indication of whether the subject required a pacemaker after the TAVR, and creating the record including the at least one parameter computed for the sample individual, and a ground truth label of the indication of whether the subject required the pacemaker after the TAVR.

Optionally, further comprising code for: in response to computing the prediction that the subject will need the pacemaker after the TAVR, generating a message including a recommendation to implant the pacemaker prior to the TAVR.

Optionally, further comprising code for implanting the pacemaker prior to the TAVR.

Optionally, the prediction is computed by a heuristic process that uses two or three of the at least one parameter.

Optionally, further comprising code for: analyzing the 3D image for identifying a first location of a perforating bundle of a conduction system of the heart and a second location of a branching point of a left bundle branch (LBB) of the conduction system, wherein the depth of the membranous septum is computed as a depth to a cardiac conduction region defined between the first location and the second location.

Optionally, further comprising code for segmenting the membranous septum, wherein the depth is computed for at least one of: a most anterior point of the membranous septum, a most posterior point of the membranous septum, a center of a line between the most anterior and most posterior points, and a center of mass of the segmented membranous septum.

Optionally, further comprising code for: computing the virtual annulus plane with respect to the 3D image, segmenting the membranous septum on the 3D image, and computing the depth as a distance from the virtual annulus plane to the segmented membranous septum.

Optionally, computing the virtual annulus comprises: identifying three nadirs of three cusps of the native aortic valve within the 3D image, and computing the virtual annulus as a plane intersecting the three nadirs.

Optionally, the rotation angle is computed along a circumference of the aorta from a point along the native aortic valve to the membranous septum.

Optionally, further comprising code for: performing a multi-series 3D imaging session, wherein a plurality of 3D images depicting at least the native aortic valve are captured at different phases of a cardiac cycle, detecting a location of at least one of the following in the plurality of 3D images depicting the different phases of the cardiac cycle: the membranous septum, commissures of the native aortic valve, nadirs of the native aortic valve, and a cardiac conduction region defined between a perforating bundle of a conduction system of the heart and a branching point of a left bundle branch (LBB) of the conduction system, computing the depth of the membranous septum and/or the angle of rotation for each of the plurality of 3D images depicting the different phases of the cardiac cycle, and computing a minimum value of the depth of the membranous and/or minimum value for the angle of rotation according to the depth and/or angle computed using the plurality of 3D images, wherein the depth of the membranous septum comprises the minimum value of the depth, and the angle of rotation comprises the minimum value of the angle.

Optionally, further comprising code for: segmenting a right ventricle endocardium (RV), a RV myocardium, a left ventricle (LV) endocardium, and a LV myocardium, identifying a region between the RV myocardium and the LV myocardium includes a minimum spatially consistent distance between the RV myocardium and the LV myocardium, wherein the depth of the membranous septum comprises the depth of the region.

Optionally, further comprising code for: detecting a posterior down slope of a notch on the LV myocardium below an aortic root, and verifying that the region corresponds to the posterior down slope of the notch of the LV myocardium.

Optionally, further comprising code for: detecting cardiac conduction region defined between a perforating bundle of a conduction system of the heart and a branching point of a left bundle branch (LBB) of the conduction system with the region, wherein the depth of the membranous septum comprises a depth of the cardiac conduction region.

According to some embodiments of the present invention, there is provided a system of training a machine learning model for predicting likelihood of a subject requiring a pacemaker after a TAVR procedure, comprising: creating a training dataset of a plurality of records of a plurality of individuals, wherein a record of an individual includes: at least one parameter computed from a sample 3D image of the individual, the at least one parameter selected from: (i) a depth of a membranous septum computed as a distance between the membranous septum and a virtual annulus plane of a native aortic valve, (ii) an angle of rotation of at least one cusp of the native aortic valve relative to the membranous septum, (iii) a left ventricle (LV)-aorta angulation, (iv) a depth of a conduction pathway computed as a distance between the conduction pathway overlying the membranous septum floor and a virtual annulus plane of a native aortic valve, wherein the conduction pathway is between a perforating bundle and a branching bundle, and a ground truth indicating whether the individual required implantation of a pacemaker after the TAVR or did not require the pacemaker, and training a machine learning model on the training dataset.

According to some embodiments of the present invention, there is provided a system of predicting likelihood of a subject requiring a pacemaker after a transcatheter aortic valve replacement (TAVR) procedure, comprising: accessing a 3D image of a subject depicting at least a native aortic valve of the subject and anatomical structures in proximity to the native aortic valve, generating a 3D model based on the 3D image of the subject, simulating physical forces applied by the 3D model on a virtual elongated tool for delivery of an aortic valve prosthesis device for implant during the TAVR procedure, computing a distance from the virtual elongated tool to a membranous septum detected on the 3D model, and computing a prediction of whether the subject will need a pacemaker after the TAVR according to the distance.

Optionally, the physical forces are applied to the virtual elongated tool by curvature of an ascending aorta, by the structure of the native aortic valve, and according to a rotation angle between at least one cusp of the native aortic valve and a membranous septum, for curving the virtual elongated tool and pushing the virtual elongated tool into a location within the native aortic valve.

Optionally, the subject is predicted to need the pacemaker when the distance is below a threshold.

According to some embodiments of the present invention, there is provided a computer implemented method of predicting likelihood of a subject requiring a pacemaker after a transcatheter aortic valve replacement (TAVR) procedure, comprising: computing from a 3D image of a subject at least one parameter selected from: (i) a distance between a conduction pathway overlying the membranous septum floor and a virtual annulus plane of a native aortic valve, wherein the conduction pathway is between a perforating bundle and a branching bundle, (ii) an angle of rotation of at least one cusp of the native aortic valve relative to the membranous septum, and (iii) a left ventricle (LV)-aorta angulation, and computing a prediction of likelihood of the subject requiring the pacemaker after the TAVR according to the at least one parameter.

According to some embodiments of the present invention, there is provided a computer implemented method of predicting likelihood of a subject requiring a pacemaker after a transcatheter aortic valve replacement (TAVR) procedure, comprising: computing from a 3D image of a subject a distance between a conduction pathway overlying the membranous septum floor and a virtual annulus plane of a native aortic valve, wherein the conduction pathway is between a perforating bundle and a branching bundle, computing a prediction of likelihood of the subject requiring the pacemaker after the TAVR according to the computed distance.

According to some embodiments of the present invention, there is provided a computer implemented method of predicting likelihood of a subject requiring a pacemaker after a transcatheter aortic valve replacement (TAVR) procedure, comprising: receiving a distance between a conduction pathway overlying the membranous septum floor and a virtual annulus plane of a native aortic valve of the subject, wherein the conduction pathway is between a perforating bundle and a branching bundle, computing a prediction of likelihood of the subject requiring the pacemaker after the TAVR according to the computed distance.

Optionally, the prediction of likelihood is computed according to a value of the depth of the conduction pathway, selected from: (i) a negative depth of the conduction pathway below zero indicating a high risk for implantation of pacemaker post TAVR, (ii) a high positive depth of the conduction pathway greater than a threshold indicating a lower risk for implantation of pacemaker post TAVR, and (iii) a low positive depth of the conduction pathway between zero and the threshold, indicating an intermediate risk for implantation of pacemaker post TAVR.

According to some embodiments of the present invention, there is provided a computer implemented method of predicting likelihood of a subject requiring a pacemaker after a transcatheter aortic valve replacement (TAVR) procedure, comprising: receiving at least one parameter of the subject selected from: (i) a depth of a membranous septum computed as a distance between the membranous septum and a virtual annulus plane of a native aortic valve, (ii) an angle of rotation of at least one cusp of the native aortic valve relative to the membranous septum, and (iii) a left ventricle (LV)-aorta angulation, and computing a prediction of likelihood of the subject requiring the pacemaker after the TAVR according to the at least one parameter.

Optionally, the angle of rotation is computed from a reference point located in the middle of the native aortic valve, by computing a first line from a point on the native aortic valve to the reference point, a second line from a location of the membranous septum to the reference point, and wherein the angle of rotation is an angle between the first line and the second line.

Optionally, the location of the membranous septum is a middle of the membranous septum.

Optionally, the point on the native aortic valve is at a commissure between a right coronary cusp (RCC) and a non-coronary cusp (NCC).

Optionally, the distance between a perforating bundle and a branching bundle overlying the membranous septum floor is used as an indicator for the location of a conduction pathway overlying the membranous septum floor, wherein the depth of the membranous septum floor is to the location of the conduction pathway.

Optionally, the at least one parameter of the subject further includes a depth of a conduction pathway computed as a distance between the conduction pathway overlying the membranous septum floor and a virtual annulus plane of a native aortic valve, wherein the conduction pathway is between a perforating bundle and a branching bundle.

Optionally, the prediction of likelihood is computed according to a value of the depth of the conduction pathway, selected from: (i) a negative depth of the conduction pathway below zero indicating a high risk for implantation of pacemaker post TAVR, (ii) a high positive depth of the conduction pathway greater than a threshold indicating a lower risk for implantation of pacemaker post TAVR, and (iii) a low positive depth of the conduction pathway between zero and the threshold, indicating an intermediate risk for implantation of pacemaker post TAVR.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of components of a system for predicting likelihood of a subject requiring a pacemaker after a TAVR procedure, in accordance with some embodiments of the present invention;

FIG. 2 is a flowchart of a method of predicting likelihood of a subject requiring a pacemaker after a TAVR procedure, in accordance with some embodiments of the present invention;

FIG. 3 includes schematics depicting features for computing the rotation angle, in accordance with some embodiments of the present invention;

FIG. 4 includes schematics different rotation angles, in accordance with some embodiments of the present invention;

FIG. 5 includes schematics different rotation angles, in accordance with some embodiments of the present invention;

FIGS. 6A-B include schematics indicating computed depth of the membranous septum relative to a virtual annulus plane, in accordance with some embodiments of the present invention;

FIG. 7 is a schematic of a 3D image including automatically identified anatomical features of a heart of a subject for computing parameters used for predicting whether the subject will require a pacemaker post TAVR, in accordance with some embodiments of the present invention;

FIG. 8 is a schematic depicting exemplary segmentations of a 3D image used for identifying a location of the membranous septum, in accordance with some embodiments of the present invention;

FIG. 9 is a schematic depicting an example of a segmentation of a myocardium of a left ventricle for identifying a location of the membranous septum, in accordance with some embodiments of the present invention;

FIG. 10 is a schematic depicting another exemplary approach for segmentation of a membranous septum from a 3D image, in accordance with some embodiments of the present invention; and

FIGS. 11A-B present results of measurements of parameters for predicting likelihood of requiring a pacemaker post TAVR obtained for multiple individuals as part of a study conducted by the Inventor, in accordance with some embodiments of the present invention.

### DETAILED DESCRIPTION

The present invention, in some embodiments thereof, relates to Transcatheter Aortic Valve Replacement (TAVR) and, more specifically, but not exclusively, to predicting whether a subject will require a pacemaker after TAVR.

As used herein, the term membranous septum and membranous septum floor (MSF) may sometimes be interchanged.

As used herein, the terms conduction pathway and cardiac conduction region are used interchangeably.

An aspect of some embodiments of the present invention relates to systems, methods, computing devices, and/or code instructions (stored on a data storage device and executable by one or more processors) for predicting likelihood of a subject requiring a pacemaker after a TAVR procedure. One or more parameters, optionally at least two parameters, are computed from a 3D image of the subject and/or are received, for example, accessed from a data storage device, obtained from a remote device, and the like. The 3D image may be, for example, a pre-procedure cardiac CT scan. A prediction of likelihood of the subject requiring the pacemaker after the TAVR is computed according to parameter(s).

The parameter(s) are selected from:
A depth of a membranous septum. The depth of the membranous septum may be computed as a distance between the membranous septum and a virtual annulus plane of a native aortic valve of the subject.
A distance between a conduction pathway overlying the membranous septum floor and a virtual annulus plane of a native aortic valve. The conduction pathway is between a perforating bundle and a branching bundle.
An angle of rotation of one or more cusps of the native aortic valve relative to the membranous septum. The angle of rotation may be between a commissure located between a right coronary cusp (RCC) and a non-coronary cusp (NCC), and the membranous septum. The angle of rotation may be along an arc on the inner wall of the aorta, as the angle between two straight lines. A first line is from the commissure to the middle of the aortic valve and a second line is from the membranous septum to the middle of the aortic valve.
A left ventricle (LV)—aorta angulation.
Indication of a right bundle branch block (RBB), which may be determined from an ECG of the subject.

Optionally, the prediction is generated by a machine learning model fed the parameter(s). The machine learning model may be trained using a supervised training approach on a training dataset of multiple records. A record includes the parameter(s) as sample input, and a ground truth indicating whether or not the subject required a pacemaker after the TAVR procedure.

Alternatively or additionally, the prediction is computed by a heuristic process and/or a set of rules. The heuristic process and/or set of rules may uses two or three or more of the parameters described herein.

Optionally, when the prediction indicates that the subject will likely require the pacemaker after the TAVR procedure, the pacemaker may be implanted during the TAVR procedure and/or in advance of the TAVR procedure.

Potential advantages of implanting the pacemaker during the TAVR procedure and/or in advance of the TAVR procedure, according to the prediction based on embodiments described herein, rather than after the TAVR procedure according to standard practice, include:
Increased safety for the patient. If the conduction system is damaged during TAVR, the patient is not left without pacing (until the pacemaker can be installed).
Increased procedure efficiency. Knowing that a pacemaker is required in advance enables better planning of both the TAVR procedure and implanting of the pacemaker.
Reduced costs by avoiding another separate procedure, such as by including the implant of the pacemaker in the same procedure as the TAVR.

An aspect of some embodiments of the present invention relates to systems, methods, computing devices, and/or code instructions (stored on a data storage device and executable by one or more processors) for predicting likelihood of a subject requiring a pacemaker after a TAVR procedure using another approach. A 3D image of a subject depicting at least a native aortic valve and anatomical structures in proximity to the native aortic valve (e.g., ascending aorta, aortic root, region of the left ventricle adjacent to the native aortic valve) is accessed. A 3D model based on the 3D image of the subject is generated. The 3D model simulates physical forces applied by the anatomy of the subject on a virtual elongated tool (e.g., catheter, guidewire) for delivery of an aortic valve prosthesis device for implant during the TAVR procedure. A distance from the virtual elongated tool to a membranous septum detected on the 3D model may be computed. The distance may vary according to the depth of the membranous septum and/or according to the angle of rotation, and/or the LV-aorta angulation. A prediction of whether the subject will need a pacemaker after the TAVR may be determined according to the simulation, optionally according to the distance.

An aspect of some embodiments of the present invention relates to systems, methods, computing devices, and/or code instructions (stored on a data storage device and executable by one or more processors) for treating a patient undergoing a TAVR procedure. A prediction of whether the subject will need a pacemaker after the TAVR procedure is determined, for example, computing using a process, obtained from a machine learning model fed one or more parameter(s) described herein, and/or a simulation, as described herein. In response to the prediction that the subject will likely need the pacemaker after the TAVR, the patient may be treated by implanting the pacemaker prior to the TAVR.

At least some embodiments described herein address the technical problem and/or medical problem of managing conduction system disturbances (e.g., arrhythmias) occurring due to a TAVR procedure. At least some embodiments described herein improve the technology and/or medical field of managing conduction system disturbances (e.g., arrhythmias) occurring due to a TAVR procedure. At least some embodiments described herein improve upon prior approaches of managing conduction system disturbances (e.g., arrhythmias) occurring due to a TAVR procedure. Trans-valvular implantation of an aortic valve prosthesis device in the aortic annulus involves manipulation of a tool such as a catheter, guidewire, and/or the aortic valve prosthesis device within the heart. Damage to the conduction system may occur as a result of the manipulation. Standard approaches to handling the conduction system disturbances are to determine the arrhythmia after the TAVR procedure, and implant a suitable pacemaker in the heart to correct the arrhythmia. Approaches to predicting whether a conduction disturbance will occur after a TAVR procedure are inaccurate, and therefore unsuitable for practical clinical use.

At least some embodiments described herein address the aforementioned technical problem(s), and/or improve the aforementioned field(s), and/or improve upon the aforementioned approach(es), by predicting likelihood of whether a subject will require a pacemaker after a TAVR procedure using one or more parameters including a depth of a membranous septum and/or an angle of rotation. Inventors discovered that a portion of the conduction system of the heart passing through the membranous septum, in particular between the perforating bundle and a branching point of a left bundle branch (LBB), is prone to damage during TAVR, leading to requiring a pacemaker. Factors which may reduce damage to the membranous septum may lead to less likelihood of requiring a pacemaker after TAVR. The depth of the membranous septum may be computed as a distance between the membranous septum and a virtual annulus plane of a native aortic valve. A larger depth may be correlated with less likelihood of damage to the conduction system requiring implantation of a pacemaker, since the membranous septum is further away from the aortic annulus where the aortic valve prosthesis device is being implanted, and therefore less likely to be damage. The angle of rotation may be of one or more cusps of the native aortic valve relative to the membranous septum. Optionally, the angle of rotation is between a commissure between a right coronary cusp (RCC) and a non-coronary cusp (NCC) and the membranous septum. Tools such as catheters and/or guidewires inserted into the left ventricle via the aorta may be “forced” into the commissure between the RCC and NCC as a result of the resiliency of the tool against the curvature of the aorta and anatomy of the aortic valve. When the angle of rotation between the commissure and the membranous septum is zero or close to zero, the tool passes by the membranous septum, which increases likelihood of damage to the membranous septum. When the angle of rotation is significantly higher than zero (positive or negative), the commissure is located further away from the membranous septum. The tools located in the commissure is positioned away from the membranous septum by the commissure, reducing likelihood of damage to the membranous septum.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

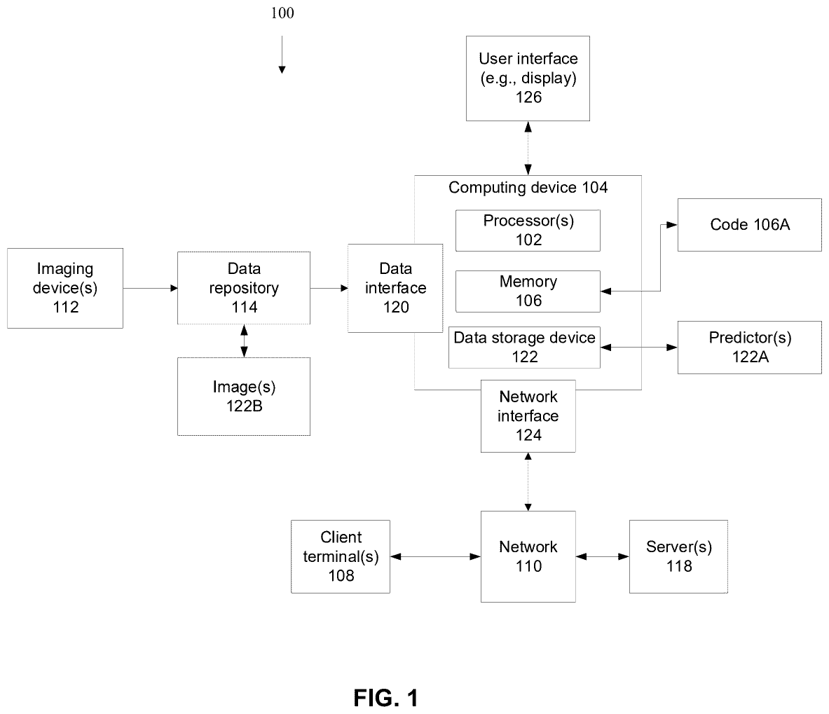

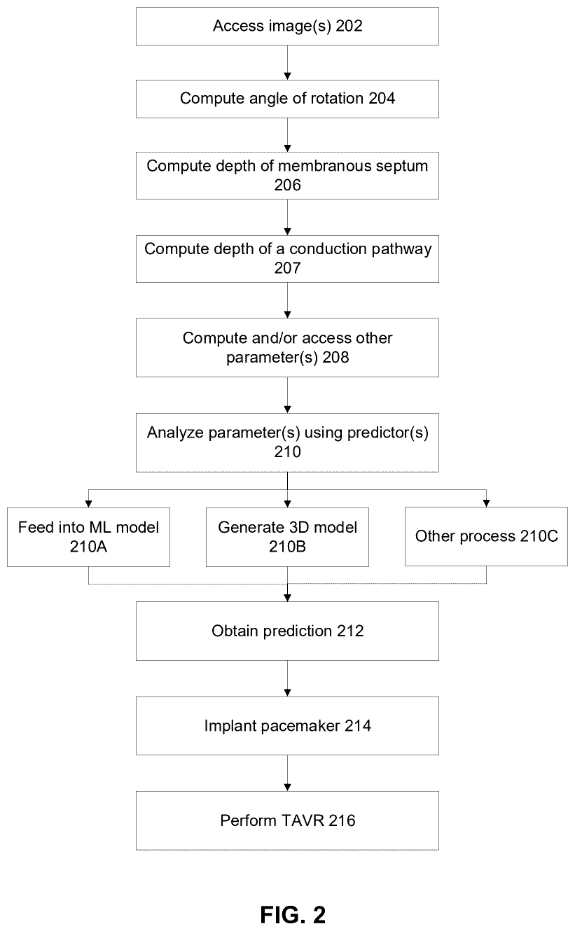

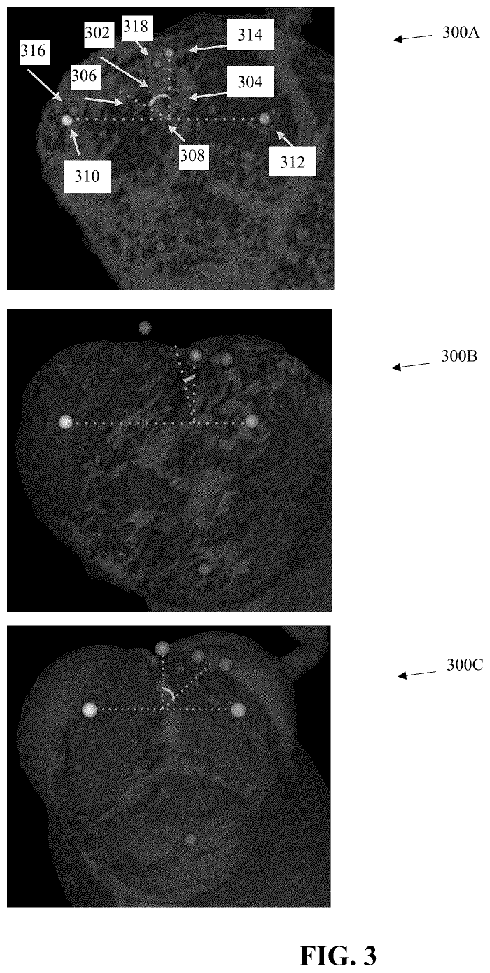

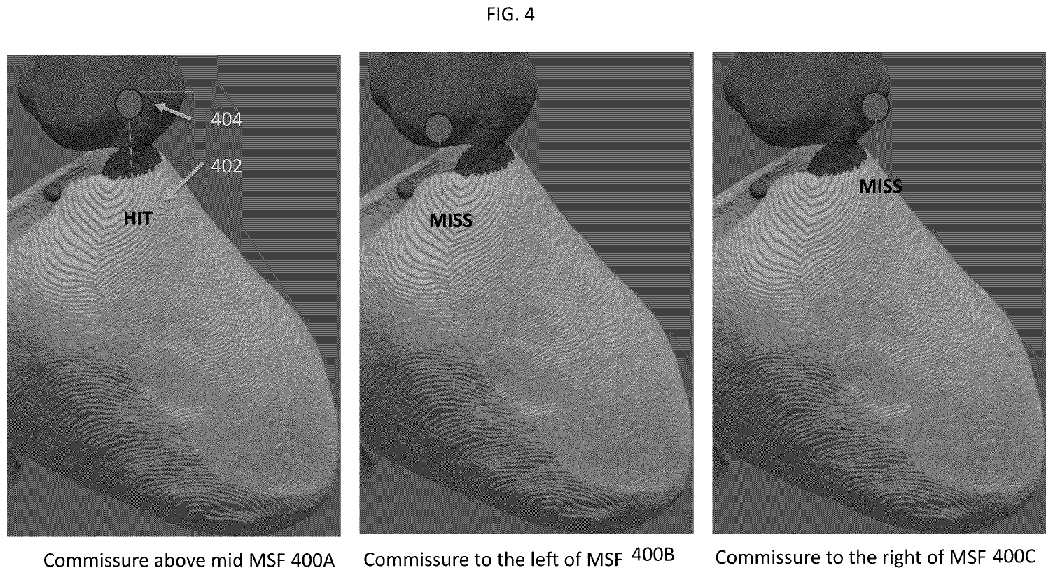

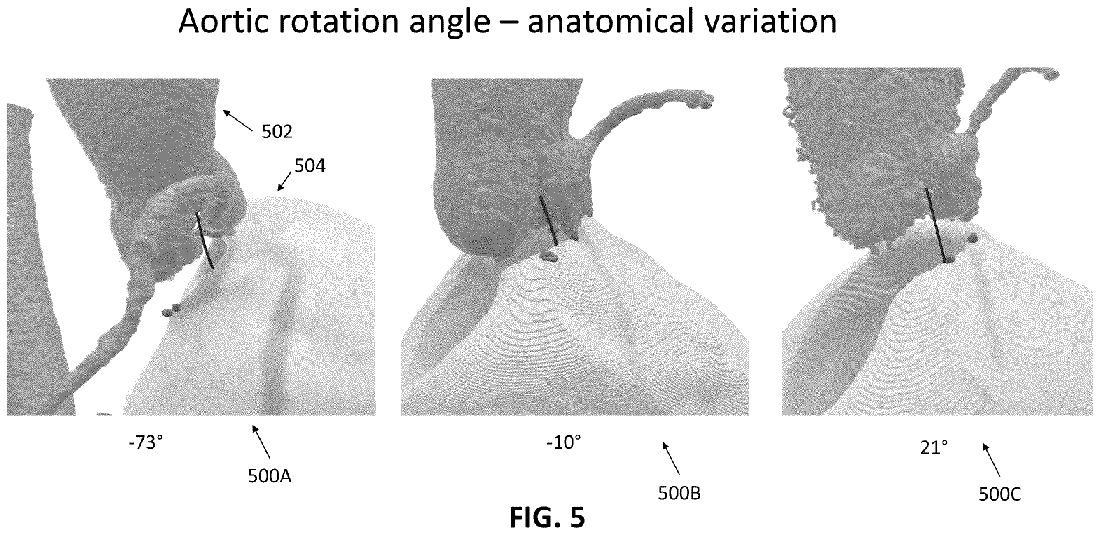

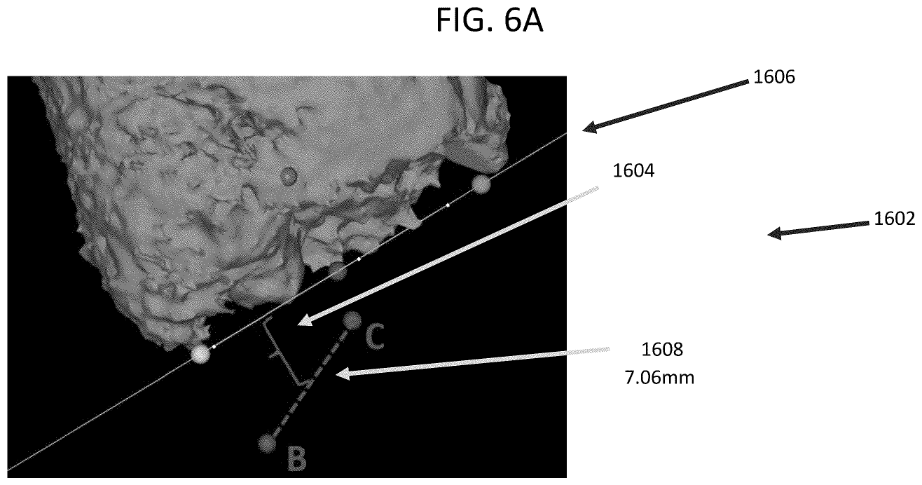

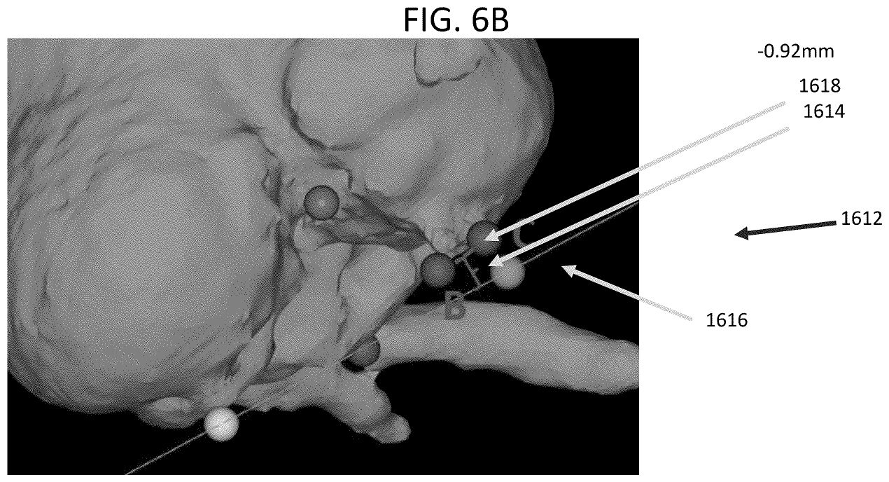

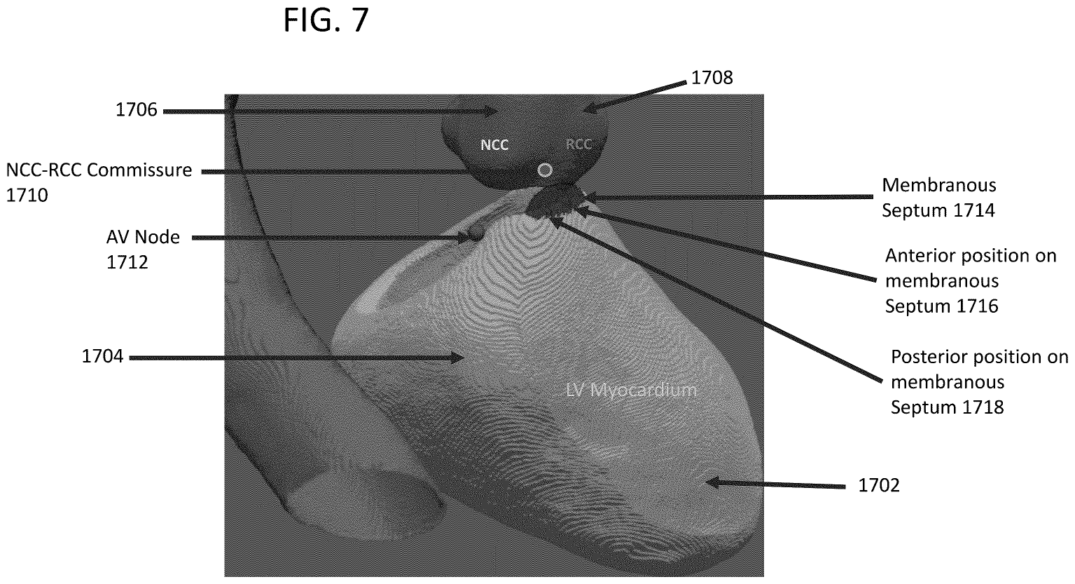

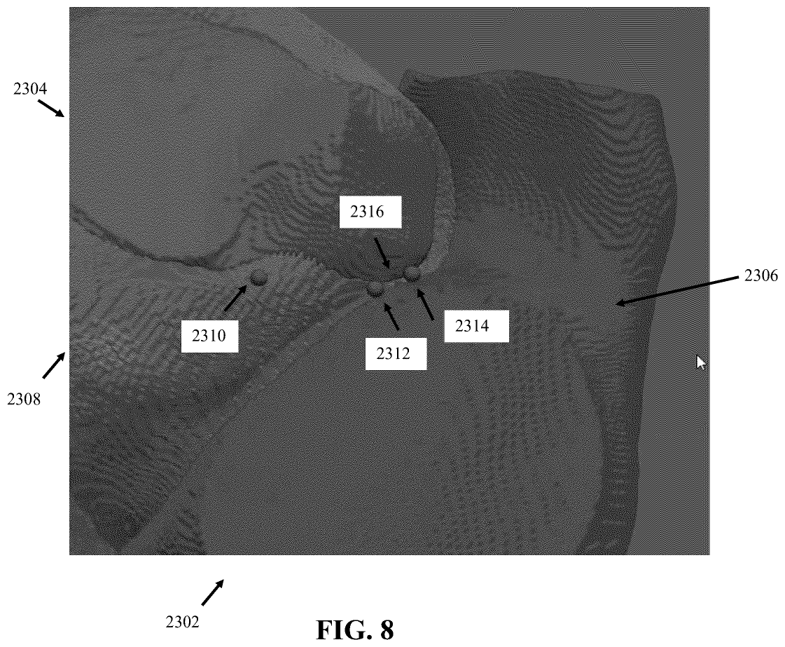

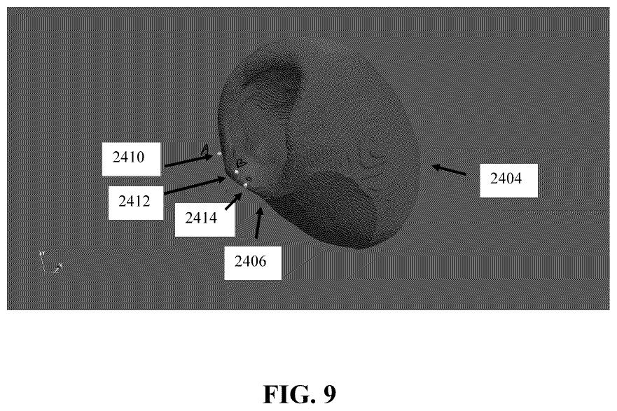

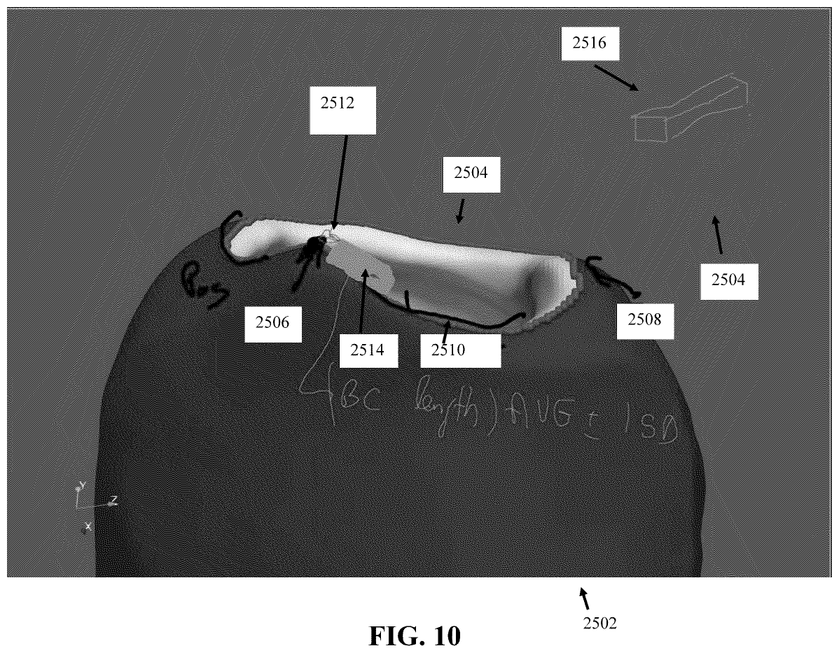

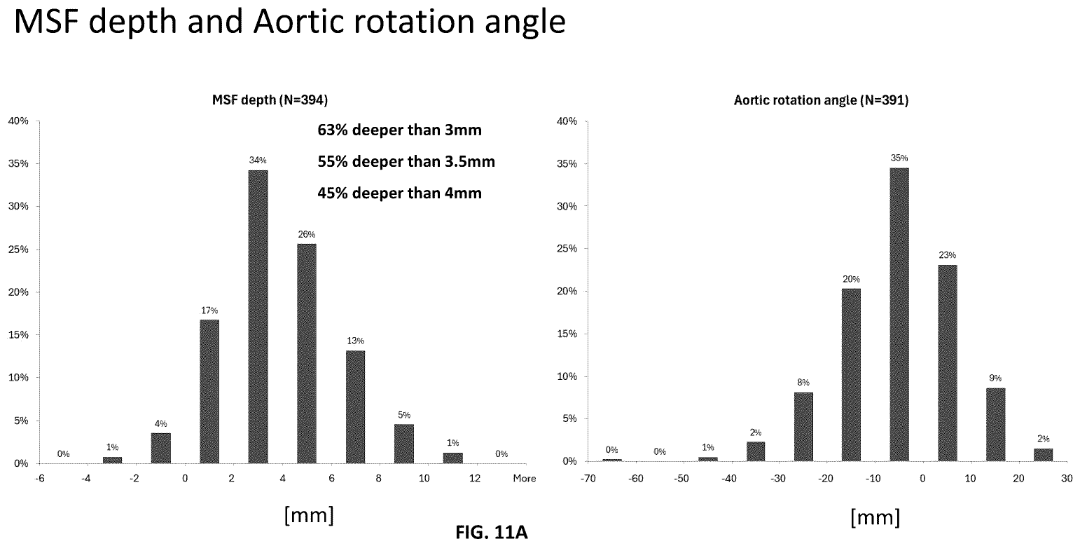

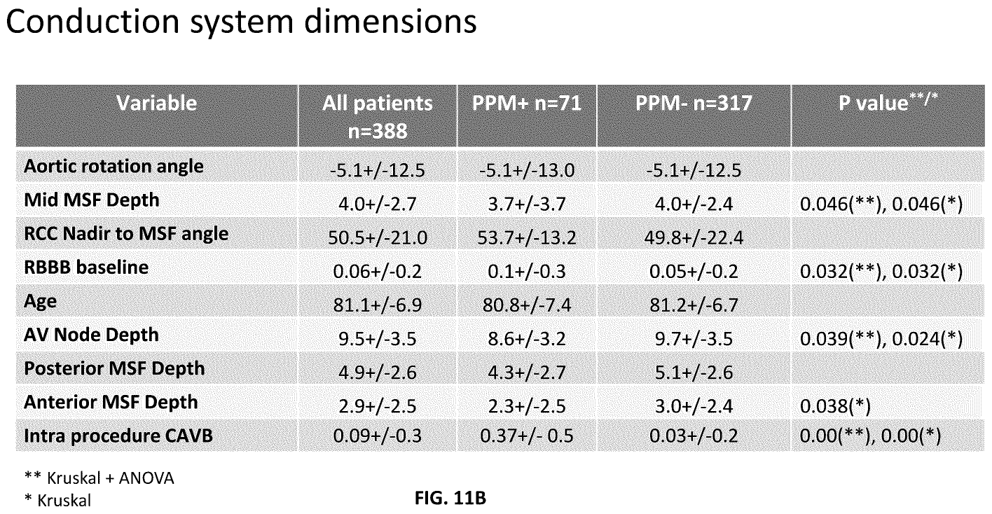

Reference is now made to FIG. 1, which is a block diagram of components of a system 100 for predicting likelihood of a subject requiring a pacemaker after a TAVR procedure, in accordance with some embodiments of the present invention. Reference is also made to FIG. 2, which is a flowchart of a method of predicting likelihood of a subject requiring a pacemaker after a TAVR procedure, in accordance with some embodiments of the present invention. Reference is also made to FIG. 3, which includes schematics depicting features for computing the rotation angle, in accordance with some embodiments of the present invention. Reference is also made to FIG. 4, which includes schematics different rotation angles, in accordance with some embodiments of the present invention. Reference is also made to FIG. 5, which includes schematics different rotation angles, in accordance with some embodiments of the present invention. Reference is also made to FIGS. 6A-B, which include schematics indicating computed depth of the membranous septum relative to a virtual annulus plane, in accordance with some embodiments of the present invention. Reference is also made to FIG. 7, which is a schematic of a 3D image including automatically identified anatomical features of a heart of a subject for computing parameters used for predicting whether the subject will require a pacemaker post TAVR, in accordance with some embodiments of the present invention. Reference is also made to FIG. 8, which is a schematic depicting exemplary segmentations of a 3D image used for identifying a location of the membranous septum, in accordance with some embodiments of the present invention. Reference is also made to FIG. 9, which is a schematic depicting an example of a segmentation of a myocardium of a left ventricle for identifying a location of the membranous septum, in accordance with some embodiments of the present invention. Reference is also made to FIG. 10, which is a schematic depicting another exemplary approach for segmentation of a membranous septum from a 3D image, in accordance with some embodiments of the present invention. Reference is also made to FIGS. 11A-B, which present results of measurements of parameters for predicting likelihood of requiring a pacemaker post TAVR obtained for multiple individuals as part of a study conducted by the Inventor, in accordance with some embodiments of the present invention.

System 100 may implement the acts of the method described with reference to FIG. 2, optionally by a hardware processor(s) 102 of a computing device 104 executing code instructions stored in a memory 106.

Computing device 104 may be implemented as, for example, a client terminal, a server, a virtual server, a radiology workstation, a catheterization laboratory workstation, a virtual machine, a computing cloud, a mobile device, a desktop computer, a thin client, a Smartphone, a Tablet computer, a laptop computer, a wearable computer, glasses computer, and a watch computer. Computing 104 may include an advanced visualization process that sometimes is add-on to a radiology and/or catheterization laboratory workstation.

Computing device 104 may include locally stored software that performs one or more of the acts described with reference to FIG. 2, and/or may act as one or more servers (e.g., network server, web server, a computing cloud, virtual server) that provides services (e.g., one or more of the acts described with reference to FIG. 2 to one or more client terminals 108 (e.g., remotely radiology workstations, remote catheterization laboratory workstations, remote picture archiving and communication system (PACS) server, remote electronic medical record (EMR) server) over a network 110, for example, providing software as a service (Saas) to the client terminal(s) 108, providing an application for local download to the client terminal(s) 108, as an add-on to a web browser and/or a medical imaging viewer application, and/or providing functions using a remote access session to the client terminals 108, such as through a web browser.

Different architectures based on system 100 may be implemented, described as follows:

In one example, computing device 104 provides centralized services. Computing device 104 may generate (e.g., perform training) of one or more predictor(s) 122A, as described herein. Alternatively, generation of predictor(s) 122A is performed by an external device, and made accessible to computing device 104 for performing the prediction. Computing device 104 may analyze locally captured images of subjects (e.g., captured by imaging device 112 such as a pre-procedure cardiac CT scan) for computation of one or more parameters (e.g., depth of membranous septum, angle of rotation), as described herein. Locally captured images may be provided to computing device 104 for centralized analysis for computation of the parameters. Images may be provided to computing device 104, for example, via an API, a local application, via a PACS server, and/or transmitted using a suitable transmission protocol. Computing device may feed the computed parameters into predictor(s) 122A to obtain the prediction of likelihood of the subject requiring a pacemaker after a TAVR procedure. The prediction may be provided, for example, to client terminal(s) 108 for presentation on a display and/or local storage, feeding into another process, stored in an electronic medical record (e.g., hosted by server 118), and/or stored by computing device 104. In another example, computing device 104 provides centralized generation of predictor(s) 122A, using images and/or ground truth (i.e., whether the patient ended up requiring a pacemaker or not after the TAVR procedure) provided by different client terminals 108 and/or servers 118. For example, different types of images may originate from different hospitals, from different physicians, for different types of aortic valve prosthesis devices, and/or for different patient population (e.g., anatomical variations, degree of aortic stenosis, and age of subject). Customized predictor(s) 122 may be generated based on the different types of images. Respective generated predictor(s) 122A may be provided to the corresponding remote devices (e.g., client terminal(s) 108 and/or server(s) 118) for local use. For example, each hospital uses the predictor created from their own images and ground truth results for predicting likelihood of pacemaker at the respective hospital.

In another example, computing device 104 provides localized services. For example, computing device 104 includes code locally stored and/or locally executed by a radiology and/or catheterization laboratory workstation, and/or client running a radiology image viewing program and/or catheterization laboratory management program. The code may be a plug-in and/or add-on to the program(s), to provide an additional feature of predicting for the subject likelihood of requiring a pacemaker after TAVR. Computing device 104 may locally create predictor(s) 122A using images 122B captured by a local imaging device 112. In another example, computing device 104 obtains predictor(s) 122A from another device. Computing device 104 computes one or more parameters by analyzing images that may be locally captured by imaging device 112, as described herein. Computing device 104 applies predictor(s) 122A to the parameter(s) for obtaining a prediction for the subject of likelihood of requiring a pacemaker after TAV. The prediction may be presented on a display (e.g., user interface 126) of computing device 104, locally stored, sent to another device for storage (e.g., PACS server), and/or fed into another application.

Imaging device 112 captures and provides the images, which are analyzed to obtain one or more parameters described herein. Imaging device 122 may be designed for capturing 3D images. Imaging device 112 may be for example, a CT machine capturing CT scans such as a cardiac CT scan, and/or an MRI machine that captures MR images. In another example, imaging device 112 may be an x-ray machine capturing x-ray images, and/or ultrasound machine capturing ultrasound images. A 3D model may be

Image(s) 122B may be stored in a data repository 114 and/or data storage device 122, for example, a storage server, a computing cloud, virtual memory, and a hard disk. Images(s) 122B may be analyzed for computing one or more parameters which are fed into and/or processed by predictor(s) 122A for generating the prediction of likelihood of a subject (depicted in the image) requiring a pacemaker post TAVR, as described herein. It is noted that image(s) 122B may be stored by a server 118, accessibly by computing device 104 over network 110.

Computing device 104 may receive image(s) 122B from imaging device 112 and/or data repository 114 using one or more data interfaces 120, for example, a wire connection (e.g., physical port), a wireless connection (e.g., antenna), a local bus, a port for connection of a data storage device, a network interface card, other physical interface implementations, and/or virtual interfaces (e.g., software interface, virtual private network (VPN) connection, application programming interface (API), software development kit (SDK)).

Hardware processor(s) 102 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 102 may include one or more processors (homogenous or heterogeneous), which may be arranged for parallel processing, as clusters and/or as one or more multi core processing units.

Memory 106 (also referred to herein as a program store, and/or data storage device) stores code instruction for execution by hardware processor(s) 102, for example, a random access memory (RAM), read-only memory (ROM), and/or a storage device, for example, non-volatile memory, magnetic media, semiconductor memory devices, hard drive, removable storage, and optical media (e.g., DVD, CD-ROM). For example, memory 106 may store image processing code 106A that implement one or more acts and/or features of the method described with reference to FIG. 2.

Computing device 104 may include a data storage device 122 for storing data, for example, one or more predictor(s) 122A as described herein. Data storage device 122 may be implemented as, for example, a memory, a local hard-drive, a removable storage device, an optical disk, a storage device, and/or as a remote server and/or computing cloud (e.g., accessed over network 110). It is noted that predictor(s) 122A may be stored in data storage device 122, with executing portions loaded into memory 106 for execution by processor(s) 102.

Computing device 104 may include a network interface 124 for connecting to network 110, for example, one or more of, a network interface card, a wireless interface to connect to a wireless network, a physical interface for connecting to a cable for network connectivity, a virtual interface implemented in software, network communication software providing higher layers of network connectivity, and/or other implementations. Computing device 104 may access one or more remote servers 118 using network 110, for example, to obtain images(s) 122B, an updated version of code 106A, and/or predictor(s) 122A.

It is noted that data interface 120 and network interface 124 may exist as two independent interfaces (e.g., two network ports), as two virtual interfaces on a common physical interface (e.g., virtual networks on a common network port), and/or integrated into a single interface (e.g., network interface). Computing device 104 may communicate using network 110 (or another communication channel, such as through a direct link (e.g., cable, wireless) and/or indirect link (e.g., via an intermediary computing device such as a server, and/or via a storage device) with one or more of:

* Client terminal(s) 108, for example, when computing device 104 acts as a server providing services (e.g., SaaS) to remote terminals, for predicting likelihood of a subject requiring a pacemaker post TAVR using predictor(s) 122A and optionally images received from respective client terminals 108.

* Server 118, for example, implemented in association with a PACS, which may store images which are analyzed for determining parameters used by the predictor(s) 122A.

* Imaging device 112 and/or data repository 114 that store images acquired by imaging device 112.

Computing device 104 and/or client terminal(s) 108 and/or server(s) 118 include and/or are in communication with a user interface(s) 126 that includes a mechanism designed for a user to enter data and/or view the prediction. Exemplary user interfaces 126 include, for example, one or more of, a touchscreen, a display, a keyboard, a mouse, and voice activated software using speakers and microphone.

Referring now back to FIG. 2, optionally at 202, one or more images of a subject are accessed. The image(s) may be 3D images and/or 2D images from which a 3D reconstruction and/or 3D model may be generated. The image may be a pre-procedure image. The image may be a CT scan, optionally a cardiac CT scan.

The image may depict at least the native aortic valve and anatomical features in proximity, including a sufficient portion of the interventricular septum expected to include the membranous septum, and optionally the aortic root and/or ascending aorta.

Alternatively to 202, the parameters described with reference to one or more of 202, 204, 206, 207, and/or 208 may be received, for example, from another executing process, from an external computing device, accessed from a data storage device, and the like.

Referring now back to FIG. 7, schematic 1702 may be computed from a 3D image of a subject, such as a pre-procedure CT scan and/or MRI, and the like. Alternatively, schematic 1702 may be computed using other approaches, such as a 3D rendering from multiple 2D images (e.g., x-rays) captured at different angles. Schematic 1702 may be, for example, one or more segmentations extracted from the 3D image, a 3D model computed from the 3D image, a 3D rendering, and image processing of the 3D image. Schematic 1702 depicts a LV myocardium 1704, NCC 1706 of the native aortic valve of the subject, and a right coronary cusp RCC 1708 of the native the aortic valve of the subject.

The following anatomical features may be automatically identified on the 3D image: NCC-RCC commissure 1710, AV node 1712, membranous septum 1714, anterior position on membranous septum (e.g., floor) 1716, and posterior position on membranous septum (e.g., floor) 1718. The membranous septum (e.g., floor) lies on the myocardium. One or more parameters for predicting whether a subject will require a pacemaker post TAVR may be computed based on the detected anatomical features, for example, angle of rotation between commissure 1710 and membranous septum 1714, and a depth of membranous septum 1714, as described herein. Since the conduction system is not shown in a CT scan, the conduction system may be computed, identified and/or imaged using known correlation between gross anatomical structures (detected from CT scan) and the conduction system. The conduction system may be computed, identified and/or imaged by a trained machine learning model and/or heuristic models. A portion of the conduction system, referred to as a cardiac conduction region, corresponding to the membranous septum may be used as a proxy to parameters computed relative to the membranous septum.

At 204, a parameter of an angle of rotation (also referred to as rotation angle) of the native aortic valve relative to the membranous septum is computed according to the image.

Exemplary approaches for detecting a location of the membranous septum, such as by segmentation, are described with reference to 206.

The angle of rotation may be computed between a region of one or more cusps and/or commissures of the native aortic valve relative to the membranous septum. Optionally, the angle of rotation may be computed between the commissure between a right coronary cusp (RCC) and a non-coronary cusp (NCC) of the native aortic valve and the membranous septum. As described herein, inventors discovered that resilient elongated tools such as a catheter and/or guidewire inserted into the left ventricle via the aorta are “pushed” into the commissure between the RCC and NCC by the curvature of the aorta and the location of the commissure. The commissure is in proximity to the membranous septum, and tools located in the commissure may damage the membranous septum.

The rotation angle may be computed along a circumference of the aorta (or a virtual circle) from the region/point along the native aortic valve to the membranous septum.

The angle of rotation may be computed from a reference point which may be defined as being located in the middle of the native aortic valve, along the circumference. The angle of rotation may be computed as the angle between two lines originating at the reference point. The first line may be computed from the point/region on the native aortic valve, optionally the commissure between the RCC and NCC to the reference point. The second line may be computed from a location on the membranous septum to the reference point.

The location of the membranous septum used to compute the angle of rotation may be a middle of the membranous septum.

Optionally, the depth of the membranous septum (e.g., as described with reference to 206) and/or the angle of rotation are computed by taking into consideration movement of the membranous septum during cardiac cycles. During beating of the heart, the membranous septum moves towards and away from the aortic valve, which dynamically changes the value of the depth of the membranous septum and/or of the angle of rotation. An exemplary process for computing the depth and/or the rotation angle in view of movement during cardiac cycles is now described. A multi-series 3D imaging session is accessed and/or performed. Multiple 3D images captured in the session depict at least the native aortic valve and the interventricular septum in proximity to the native aortic valve which is expected to include the membranous septum. The multiple 3D image are captured at different phases of a cardiac cycle. A location of one or more the following is detected in each one of the multiple 3D images depicting the different phases of the cardiac cycle: the membranous septum, commissures of the native aortic valve, nadirs of the native aortic valve, and/or the cardiac conduction region (defined between the perforating bundle and the branching point of the LBB). The depth of the membranous septum and/or the angle of rotation may be computed for each of the 3D images depicting the different phases of the cardiac cycle. The depth of the membranous septum and/or the angle of rotation may be defined as a minimum value of the depth of the membranous and/or a minimum value for the angle of rotation computed over the multiple 3D images.

Referring now back to FIG. 3, schematics 300A-C depict features for computing the rotation angle, as described herein. Schematic 300A depicts a rotation angle of −47.3 degrees, schematic 300B depicts a rotation angle of −10 degrees, and schematic 300C depicts a rotation angle of +33.6 degrees.

Features for computing the rotation angle numbered on schematic 300A have corresponding features on schematics 300B and 300C (but are not numbered for clarity). Schematic 300A may be obtained from a CT scan of a subject. Angle 302 may be computed as the angle between a line 304 and a line 306. Both lines 304 and 306 originate from a common origin 308, which may be, for example, a center of the aortic valve, and/or located midway between a nadir of the NCC 310 and a nadir of the RCC 312. A nadir of a left coronary cusp (LCC) 314 is shown for completion. Line 304 may be between original 308 and a commissure 314 between the RCC and NCC. Line 306 may be between origin 308 and a midpoint of a membranous septum which may be defined as the midpoint between the branching point of the LBB 316 and a perforating bundle 318 and/or between two ends of the membranous septum (also indicated by 316 and 318).

Referring now back to FIG. 4, schematics 400A-C depict different rotation angles. Features for computing the rotation angle numbered on schematic 400A have corresponding features on schematics 400B and 400C (but are not numbered for clarity). In schematic 400A a membranous septum 402 is aligned with a commissure 404 between the NCC and RCC, indicating an angle of zero or close to zero. In schematic 400B, the commissure is to the left of the membranous septum, which may indicate a positive (negative) angle of rotation. In schematic 400C, the commissure is to the right of the membranous septum, which may indicate a negative (positive) angle of rotation.

Referring now back to FIG. 5, schematics 500A-C depict different rotation angles. Schematic 500A depicts a rotation angle of −73 degrees, schematic 300B depicts a rotation angle of −10 degrees, and schematic 300C depicts a rotation angle of +21 degrees.

Features for computing the rotation angle numbered on schematic 500A have corresponding features on schematics 500B and 500C (but are not numbered for clarity). In schematic 500A an aortic root 502 and a left ventricle 504 are shown, for example, segmented from a CT scan. The angle of rotation is computed by identifying features such as the commissure between the RCC and NCC on aortic root 502 and location of the membranous septum on left ventricle 504, for example, as described herein.

At 206, one or more parameters relate to a membranous septum are computed. The parameter(s) may indicate a depth of the membranous septum. The depth of the membranous septum may be computed as a distance between the membranous septum and a virtual annulus plane of a native aortic valve of the subject.

Inventors discovered that a pacemaker may be required after a TAVR procedure when the cardiac conduction region running through the membranous septum is damaged due to manipulation of tools during the TAVR procedure. The cardiac conduction region running through the membranous septum is especially vulnerable to be damaged during the TAVR procedure due to its location close to the surface of the membranous septum.

The parameter may relate to a depth of the location of the cardiac conduction region within the membranous septum. The cardiac conduction region may serve as a proxy to the membranous septum in parameters described herein, for example, the depth of the cardiac conduction region is a proxy to the depth of the membranous septum, and/or angle of rotation between the cardiac conduction region and the commissure between the NCC and RCC is a proxy to the angle of rotation between the membranous septum and the commissure. The 3D image may be analyzed for identifying the cardiac conduction region defined between a first location and a second location. The first location may be of a perforating bundle. The second location may be of a branching point of the LBB. The location of the cardiac conduction region, which may be overlying the membranous septum floor, may be defined as between the first location of perforating bundle and the second location of the branching point of the LBB.

The depth of the membranous septum may be defined to a region/point on the membranous septum, for example, at least one of: a most anterior point of the membranous septum, a most posterior point of the membranous septum, a center of a line between the most anterior and most posterior points, and a center of mass of the segmented membranous septum.

The computed depth of the membranous septum may be presented on the display.

The virtual annulus plane may be computed according to the 3D image. The virtual annulus plane may be computed by identifying three nadirs of three cusps of the native aortic valve within the 3D image, and computing the virtual annulus as a plane intersecting the three nadirs.

Some features described herein with reference to the depth of the membranous septum may refer to the distance between the conduction pathway and the virtual annulus plane (e.g., as described with reference to 207).

Referring now back to FIGS. 6A-6B, schematics 1602 and 1612 indicate a computed membranous septum depth, computed as a distance between a membranous septum to a virtual annulus plane. Schematic 1602 depicts a membranous septum depth 1604 of 7.06 millimeters (mm) (i.e., a positive depth). Membranous septum depth 1604 is defined between a virtual annulus plane (shown as a line) 1606 and membranous septum 1608. Annulus line 1606 may be defined as a plane/line that inserts three nadirs of the leaflets of the aortic valve. Membranous septum depth 1608 may be defined as a line between the perforating bundle the branching point of the LBB, as described herein), and/or between an anterior position on the membranous septum and a posterior position on the membranous septum. Schematic 1612 depicts a membranous septum depth 1614 of −0.92 mm (i.e., a negative depth). It is noted that in schematic 1612, annulus line 1616 is below membranous septum 1618, in contrast to schematic 1602 where annulus line 1606 is above membranous septum 1608.

The membranous septum may be segmented from the 3D image. Segmenting/detecting the membranous septum is technically challenging due to the small size of the membranous septum and/or difficulty in visualizing the membranous septum in medical images. Exemplary approaches for segmenting/detecting the membranous septum are now provided.

One exemplary approach for automatic segmentation of the membranous septum from a 3D image is now described. The exemplary approach is based on segmenting the posterior border of the membranous septum and the anterior border (e.g., point) of the membranous septum. The AV node may not necessarily may be segmented.

The membranous septum sits between the tip of the septal muscle and connects to the aortic root. The floor of the membranous septum includes part of the conduction axis (His Bundle and/or perforating bundle).

The AV node may be segmented using the following exemplary approach: Identification of both hinges of the anterior cusps of the aortic valve and posterior cusps of the aortic valve, in a two chamber and/or three chamber view in NPR. Adjusting the parallel slice planes of the 3D image (e.g., CT scan that includes the parallel slice planes) to the superior edge of the hinges. Locating an area between the medial commissure of the mitral valve and the right atrium, optionally in a short axis view. The AV node may be annotated in close proximity to the atrial wall or the apex of the inferior pyramidal space (if clearly visible).

The posterior border of the floor of the membranous septum may be segmented using the following exemplary approach: Identification of the aortic annulus plane, such as by intersecting nadirs of the three cusps of the aortic valve (i.e., NCC, LCC, and RCC). Identification of the membranous septum posterior border by identification of the roof of the inferoseptal recess in the short axis plane. Identification and optional annotation of an inferior area of the membranous septum adjacent to the roof of the inferoseptal recess, optionally in a long axis view.

The anterior point of the floor of the membranous septum may be segmented using the following exemplary approach: Performing clockwise rotation of each slice plane of the 3D image, optionally in a short axis, until the anterior edge of the membranous septum is identified, optionally in a long axis view. Annotation of a point between the inferior area of the membranous septum and the crest of the septum, optionally in the long axis view.

Another exemplary approach for automatic segmentation of the membranous septum from a 3D image is now described. The 3D image may be a contrast enhanced cardiac CT scan. The 3D image may be segmented to identify the left and the right ventricles and the muscular ventricular septum between the ventricles. A tip of the muscular ventricular septum is identified and optionally marked for each slice of multiple slices of the 3D image, such as CT scan. A line connecting the superior point of the ventricular septal muscle through all the slices may be defined as the membranous septum floor. A sub-region within the line that includes the His bundle is identified within the membranous septum floor. The sub-segment may be defined between a first inferior about 10% or 20% or other percentage of the membranous septum floor to an end of the cardiac conduction region located at about 70%, or 80%, or 85%, or 90% of the length of the membranous septum floor.

Referring now back to FIG. 8, schematic 2302 depicts exemplary segmentations of a 3D image used for identifying the membranous septum. The location of the membranous septum is used to compute one or more parameters described herein used for predicting likelihood of a patient requiring a pacemaker post TAVR. Segmentation 2304 represents the endocardium of the left ventricle. Segmentation 2306 represents the right ventricle, in particular, the endocardium. Segmentation 2308 represents the myocardium of the left ventricle. AV node 2310, penetrating bundle 2312, and branching point of the LBB 2314 may be identified. The membranous septum may be defined as a region 2316 between penetrating bundle 2312 and branching point of the LBB 2314, where segmentation 2308 is minimal between segmentation 2304 and segmentation 2306, and is in the posterior slope of the LV myocardium notch shown with reference to FIG. 9. 2316 may represent an area of apposition of the right ventricle (RV) and the left ventricle (LV).

An exemplary approach for segmentation of the membranous septum is as follows: identifying a region between the RV myocardium segmentation and the LV myocardium segmentation that includes a minimum spatially consistent distance between the RV myocardium and the LV myocardium, is identified. A length of a base of the region may be, for example, greater than about 0.8 mm, or 1 mm, or 1.2 mm, and/or shorter than about 10 mm, or 12 mm, or 14 mm, or other values. The thickness of membranous septum may be on the lower (possibly the lowest) percentile of distance between the segmentations of the LV and RV. Identifying an area defined as the edge of the LV myocardial segmentation that corresponds to the region of the left and right ventricles segmentation. Verifying that the identified area substantially matches a posterior down slope, optionally the notch, of the LV myocardium segmentation. Marking the path of the membranous septum floor that is included in the region of the LV myocardium segmentation apposition of the right ventricle to the left ventricle. Marking on the identified area the posterior down slope of left ventricle myocardium segmentation.

Referring now back to FIG. 9, the segmentation of the myocardium of left ventricle 2404 from a 3D image may is presented, optionally on a display. A notch in the left ventricle wall is indicated by dashed line 2406. The notch may be on the left ventricle myocardium segmentation, below the aortic root. The posterior down slope of the notch may be marked, for example, using a straight line with thickness and/or using a thick line (e.g., thickness is a function of the CT resolution and quality). AV node 2410, penetrating bundle 2412, and branching point of the LBB 2414, are shown. The membranous septum may be identified as being located between penetrating bundle 2412 and branching point of the LBB 2414.

Referring now back to FIG. 10, schematic 2502 depicts another exemplary approach for segmentation of the membranous septum from a 3D image. A left ventricle myocardium base rim 2504 is identified. A first point 2506 and a second point 2508 are identified. An anterior myocardial notch 2510 is defined as passing between points 2506 and 2508. A space 2512 apart from first point 2506 is used to define a start of a cardiac conduction region 2514 between a perforating bundle and a branching point of the LBB. The cardiac conduction region 2514 indicates the location of the membranous septum. Space 2512 may be, for example, about 0.8 mm, or 1 mm, or 1.2 mm, or other values. A box 2516 is defined. The dimensions of box 2516 may be a predefined width (e.g., about 0.8 mm, or 1 mm, or 1.2 mm), a predefined height (e.g., about 0.8 mm, or 1 mm, or 1.2 mm) and a length. The length may be computed as a predicted length of the cardiac conduction region with margin of error, for example, average of multiple subjects plus a standard deviation (e.g., about 0.5, 1, 1.5, or 2 standard deviations). Box 2516 may be positioned parallel to and a preselected distance (e.g., above 0.3 mm, or 0.5 mm, or 0.7 mm) above the left ventricle myocardial base rim. Box 2516 defines the location of the membranous septum.

At 207, a distance between a conduction pathway (optionally overlying the membranous septum floor) and the virtual annulus plane of the native aortic valve, also referred to herein as a depth of the conduction pathway, may be computed. The conduction pathway described herein is defined as being located between a perforating bundle and a branching bundle, optionally the LBB.

The computed depth of the conduction pathway may be presented on the display.

Inventor discovered that the depth of the conduction pathway may be divided into three ranges, which may be separated by thresholds:
A negative depth of the conduction pathway, i.e., where the conduction pathway is located superior to the annulus (i.e., closer to the head). For example, depth of the conduction pathway is less than zero. The negative depth of the conduction pathway may indicate a high risk for implantation of pacemaker post TAVR (e.g., high risk of damage to the conduction pathway occurring during the procedure).
A high positive depth of the conduction pathway, i.e., where the conduction pathway is located inferior to the annulus (i.e., further away from the head). For example, depth of the conduction pathway is greater than a threshold, for example, greater than about 4 mm, or 5 mm, or 6, mm, or 7 mm, or 8 mm, or 9 mm, or 10 mm, or greater. The high positive depth of the conduction pathway may indicate a lower risk for implantation of pacemaker post TAVR (e.g., low risk of damage to the conduction pathway occurring during the procedure).
A low positive depth of the conduction pathway. For example, depth of the conduction pathway is between zero and the threshold, for example, about 0-3 mm, or 0-4 mm, or 0-5 mm, or other ranges. The low positive depth of the conduction pathway may indicate an intermediate risk for implantation of pacemaker post TAVR (e.g., medium risk of damage to the conduction pathway occurring during the procedure).

At 208, one or more other parameters may be obtained and/or computed. Examples of other parameters include:
A left ventricle (LV)—aorta angulation. The LV-aorta angulation may be calculated from a CT angiography image, from an implantation projection in which the three cusps of the aortic valve are aligned. The LV-aorta angulation may be defined as the angle between a horizontal plane and a plane of the aortic annulus. A profile of a centerline may be exported from a software package as a set of points defined by 3D coordinates which may be stored in a file with extensible markup language (xml) format.
Other parameters of the conduction system, which may be measurements of the conduction system, for example, mid MSF depth, RCC nadir to MSF angle, AV node depth, posterior MSF depth, anterior MSF depth, penetrating bundle depth, and branching bundle depth.
Whether the subject is experiencing a right bundle branch block. The diagnosis of the right bundle branch block may be obtained, for example, by analyzing an ECG (manually and/or automatically), and/or from a medical record of the subject.
Parameters of the aortic valve prosthesis device which is to be deployed, for example, type, manufacturer, length and/or diameter which vary from initial crimp diameter to final expanded diameter, and the like.
Parameters of other tools used in the TAVR procedure, for example, type of guidewire, resiliency of guidewire (e.g., soft, hard), diameter of guidewire, type of catheter, and the like.
Parameter of the subject, for example, degree of aortic stenosis, weight, age, measurements of anatomical landmarks in the heart, and the like.

At 210, a prediction of likelihood of the subject requiring the pacemaker after the TAVR is computed according to one or more parameters described herein.

Optionally, the prediction is based on the angle of rotation.

Alternatively, the prediction is based on the depth of the membranous septum.

Alternatively, the prediction is based on a combination of the depth of the membranous septum and the angle of rotation.

Alternatively, the prediction is based on a combination of the depth of the membranous septum and the angle of rotation and one or more parameters described with reference to 208.

Different exemplary predictors for computing the prediction are described with reference to 210A-C.

At 210A, the predictor may be implemented as a machine learning model. The prediction may be computed by feeding the parameter(s) into the machine learning model, and obtain the prediction generated by the machine learning model.

The machine learning model may be implemented using a suitable architecture for processing the parameters, for example, a binary classifier, a multi-class classifier, a statistical classifier, one or more neural networks of various architectures (e.g., convolutional, fully connected, deep, encoder-decoder, recurrent, transformer, graph, combination of multiple architectures), support vector machines (SVM), logistic regression, k-nearest neighbor, decision trees, boosting, random forest, a regressor and the like.

The machine learning model may be trained using a supervised approach on a training set of records of multiple sample individual. Each record includes one or more parameters described herein computed from a sample 3D image(s) of the sample individual and/or other parameters obtained using other approaches (e.g., from the electronic medical record). Each record is labelled with ground truth indicating whether the subject required a pacemaker after the TAVR or did not require the pacemaker.

Optionally, when the subject did require a pacemaker, the type of conduction disturbance (e.g., arrhythmia) which the pacemaker was inserted to treat is indicated. When a pacemaker is predicted to be required, the type of conduction disturbance to treat may be predicted.

Alternatively or additionally, at 210B, the predictor is implemented as a 3D model that simulates one or more features of the TAVR procedure for predicting damage to the conduction system which require treatment by implantation of a pacemaker.

The 3D model may be created based on the 3D image of the subject and/or according to one or more parameters described herein. The 3D model may be created by segmenting one or more of: the ascending aorta, the aortic root, the native aortic valve, cusps of the native aortic valve, the membranous septum, region of the intraventricular septum expected to include the membranous septum, and/or left ventricle.

The 3D model simulates physical forces based on the parameter(s) and/or other anatomical features such as curvature of the aorta, on a virtual elongated tool expected to be using during the TAVR procedure. The virtual elongated tool may be modeled as resilient, having its shape modified according to the curvature of the ascending aorta and/or anatomy of the heart defined by the parameter(s) such as the angle of rotation and/or depth of the membranous septum. A path of the virtual elongated tool inserted into the left ventricle from the aorta, may be simulated according to the parameter(s). The 3D model simulates curvature of the virtual elongated tool and the “pushing” of the virtual elongated tool into a location within the native aortic valve, such as the commissure between the NCC and RCC. The virtual elongated tool may simulate, for example, a guidewire and/or a catheter for delivering and deploying the aortic valve prosthesis device. The 3D model may simulate deployment of the aortic valve prosthesis device within the native aortic valve.

The 3D model may be analyze to compute a distance from the virtual elongated tool, optionally while it is located in the commissure between the NCC and RCC, to the membranous septum. The prediction may be based on the distance. A closer distance may be correlated with a higher risk of requiring a pacemaker prior to TAVR. A farther distance may be correlated with a lower risk of requiring the pacemaker prior to TAVR. The subject may be predicted to need the pacemaker when the distance is below a threshold.

Alternatively or additionally, at 210C, the prediction is computed by a heuristic process and/or a set of rules. The heuristic process and/or set of rules may uses two or three or more of the parameters described herein.

At 212, a prediction of likelihood of the subject requiring the pacemaker after a transcatheter aortic valve replacement (TAVR) procedure is obtained from the predictor.

The prediction may be a binary classification, such as between a first category indicating that the subject requires a pacemaker and a second category indicating that the subject does not require a pacemaker. In another example, the prediction may be, for example, high risk of requiring a pacemaker (e.g., post procedure and/or high risk of damage to the cardiac conduction system during the procedure) or low risk. In yet another example, the prediction may be, for example, high risk of damage to the cardiac conduction system indicating a recommendation for implantation of the pacemaker prior to the procedure, and low risk of damage to the cardiac conduction system which does not justify pre-procedure implantation of the pacemaker.

In yet another implementation, the classification may be into three or more classification categories, for example, low risk, medium risk, and high risk.

The classification into categories may be performed, for example, by comparing the predicted likelihood to one or more thresholds and/or ranges defining the categories. For example, a predicted value from 0-0.2 may be defined as low risk, from 0.2-0.8 as medium risk, and from 0.8-1.0 as high risk. Alternatively, the classification into categories may be directly performed, optionally by a classifier (or other machine learning model), which may be trained on a training dataset of records including ground truth labels of the different categories.

Alternatively or additionally, the prediction includes a percentage of likelihood (i.e., probability) that the subject will require a pacemaker. Subjects with probability percentages above a threshold may be selected for advanced implant of the TAVR, for example, above about 70%, or 80%, or 90%, or other thresholds.

Optionally, the prediction is provided, for example, to a client terminal (e.g., a message presented on a display), to a storage device (e.g., stored in an electronic medical record of the subject), and the like.

Optionally, in response to computing the prediction that the subject will need the pacemaker after the TAVR, a message including a recommendation to implant the pacemaker prior to the TAVR is generated and presented, for example, on a display of a computed when the physician assigned to treat the subject logs in.

At 214, the pacemaker may be implanted prior to the TAVR and/or during the TAVR, rather than after the TAVR once a conduction disturbance has been determined.

At 216, the TAVR procedure may be conducted in the patient, in which the pacemaker has already been implanted in advance of the TAVR procedure and/or during the TAVR procedure according to the prediction.

Various embodiments and aspects of the present invention as delineated hereinabove and as claimed in the claims section below find experimental and/or support in the following examples.

### EXAMPLES

Reference is now made to the following examples, which together with the above descriptions illustrate some embodiments of the invention in a non limiting fashion.

Inventors conducted a study, in which one or more of the parameters described herein were computed and/or measured for multiple subjects. An indication of whether each subject required a pacemaker after TAVR may be obtained, and used as ground truth associated with the parameters for generating one or more predictors, for example, for generating a training dataset for training a machine learning model, for creating a simulation model, and/or for creating a heuristic approach, as described herein.

Referring now back to FIGS. 11A-B, results of measurements of parameters for predicting likelihood of requiring a pacemaker post TAVR obtained for multiple individuals as part of a study conducted by the Inventor, are presented.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant aortic valve prosthesis devices will be developed and the scope of the term aortic valve prosthesis device is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A computer implemented method of predicting likelihood of a subject requiring a pacemaker after a transcatheter aortic valve replacement (TAVR) procedure, comprising:
computing from a 3D image of a subject at least one parameter selected from:
(i) a depth of a membranous septum computed as a distance between the membranous septum and a virtual annulus plane of a native aortic valve,
(ii) an angle of rotation of at least one cusp of the native aortic valve relative to the membranous septum, and
(iii) a left ventricle (LV)-aorta angulation; and
computing a prediction of likelihood of the subject requiring the pacemaker after the TAVR according to the at least one parameter.

2. The computer implemented method of claim 1, wherein the angle of rotation is computed from a reference point located in the middle of the native aortic valve, by computing a first line from a point on the native aortic valve to the reference point, a second line from a location of the membranous septum to the reference point, and wherein the angle of rotation is an angle between the first line and the second line.

3. The computer implemented method of claim 2, wherein the location of the membranous septum is a middle of the membranous septum.

4. The computer implemented method of claim 2, wherein the point on the native aortic valve is at a commissure between a right coronary cusp (RCC) and a non-coronary cusp (NCC).

5. The computer implemented method of claim 1, wherein the distance between a perforating bundle and a branching bundle overlying the membranous septum floor is used as an indicator for the location of a conduction pathway overlying the membranous septum floor, wherein the depth of the membranous septum floor is to the location of the conduction pathway.

6. The computer implemented method of claim 1, wherein the at least one parameter further includes a depth of a conduction pathway computed as a distance between the conduction pathway overlying the membranous septum floor and a virtual annulus plane of a native aortic valve, wherein the conduction pathway is between a perforating bundle and a branching bundle.

7. The computer implemented method of claim 6, wherein the prediction of likelihood is computed according to a value of the depth of the conduction pathway, selected from:
(i) a negative depth of the conduction pathway below zero indicating a high risk for implantation of pacemaker post TAVR,
(ii) a high positive depth of the conduction pathway greater than a threshold indicating a lower risk for implantation of pacemaker post TAVR, and
(iii) a low positive depth of the conduction pathway between zero and the threshold, indicating an intermediate risk for implantation of pacemaker post TAVR.

8. The computer implemented method of claim 1, wherein the at least one parameter further includes an indication of whether the subject is experiencing a right bundle branch block.

9. The computer implemented method of claim 1, further comprising generating a 3D model based on the 3D image of the subject and according to the at least one parameter, the 3D model simulating physical forces applied according to the at least one parameter on a virtual elongated tool for delivery of an aortic valve prosthesis device for implant during the TAVR procedure, further comprising analyzing the 3D model to compute a distance from the virtual elongated tool to the membranous septum, wherein the prediction is based on the distance.

10. The computer implemented method of claim 9, wherein the physical forces are applied to the virtual elongated tool by curvature of an ascending aorta, and according to the anatomy of the heart defined by the at least one parameter, for curving the virtual elongated tool and pushing the virtual elongated tool into a location within the native aortic valve.

11. The computer implemented method of claim 9, wherein a path of the virtual elongated tool inserted into a left ventricle from the aorta, is computed using the at least one parameter.

12. The computer implemented method of claim 11, wherein the path of the virtual elongated tool inserted into the left ventricle from the aorta is computed, is used to predict the interaction of the tool with the membranous septum.

13. The computer implemented method of claim 1, wherein computing the prediction comprises feeding the at least one parameter into a machine learning model, and obtaining the prediction from the machine learning model.

14. The computer implemented method of claim 13, wherein the machine learning model is trained on a plurality of records created for a plurality of sample individuals, wherein a record for a sample individual created is created by:
computing the at least one parameter from a sample 3D image of the sample individual;
accessing an indication of whether the subject required a pacemaker after the TAVR; and
creating the record including the at least one parameter computed for the sample individual, and a ground truth label of the indication of whether the subject required the pacemaker after the TAVR.

15. The computer implemented method of claim 1, further comprising: in response to computing the prediction that the subject will need the pacemaker after the TAVR, generating a message including a recommendation to implant the pacemaker prior to the TAVR and/or implanting the pacemaker prior to the TAVR.

16. The computer implemented method of claim 1, wherein the prediction is computed by a heuristic process that uses two or three of the at least one parameter.

17. The computer implemented method of claim 1, further comprising:
analyzing the 3D image for identifying a first location of a perforating bundle of a conduction system of the heart and a second location of a branching point of a left bundle branch (LBB) of the conduction system;
wherein the depth of the membranous septum is computed as a depth to a cardiac conduction region defined between the first location and the second location.

18. The computer implemented method of claim 1, further comprising segmenting the membranous septum, wherein the depth is computed for at least one of: a most anterior point of the membranous septum, a most posterior point of the membranous septum, a center of a line between the most anterior and most posterior points, and a center of mass of the segmented membranous septum.

19. The computer implemented method of claim 1, further comprising:
computing the virtual annulus plane with respect to the 3D image;
segmenting the membranous septum on the 3D image; and
computing the depth as a distance from the virtual annulus plane to the segmented membranous septum.

20. The computer implemented method of claim 18, wherein computing the virtual annulus comprises: identifying three nadirs of three cusps of the native aortic valve within the 3D image, and computing the virtual annulus as a plane intersecting the three nadirs.

21. The computer implemented method of claim 1, wherein the rotation angle is computed along a circumference of the aorta from a point along the native aortic valve to the membranous septum.

22. The computer implemented method of claim 1, further comprising:
performing a multi-series 3D imaging session, wherein a plurality of 3D images depicting at least the native aortic valve are captured at different phases of a cardiac cycle;
detecting a location of at least one of the following in the plurality of 3D images depicting the different phases of the cardiac cycle: the membranous septum, commissures of the native aortic valve, nadirs of the native aortic valve, and a cardiac conduction region defined between a perforating bundle of a conduction system of the heart and a branching point of a left bundle branch (LBB) of the conduction system;
computing the depth of the membranous septum and/or the angle of rotation for each of the plurality of 3D images depicting the different phases of the cardiac cycle; and
computing a minimum value of the depth of the membranous and/or minimum value for the angle of rotation according to the depth and/or angle computed using the plurality of 3D images,
wherein the depth of the membranous septum comprises the minimum value of the depth, and the angle of rotation comprises the minimum value of the angle.

23. The computer implemented method of claim 1, further comprising:
segmenting a right ventricle endocardium (RV), a RV myocardium, a left ventricle (LV) endocardium, and a LV myocardium;
identifying a region between the RV myocardium and the LV myocardium includes a minimum spatially consistent distance between the RV myocardium and the LV myocardium,
wherein the depth of the membranous septum comprises the depth of the region.

24. The computer implemented method of claim 23, further comprising:
detecting a posterior down slope of a notch on the LV myocardium below an aortic root; and
verifying that the region corresponds to the posterior down slope of the notch of the LV myocardium.

25. The computer implemented method of claim 23, further comprising:
detecting cardiac conduction region defined between a perforating bundle of a conduction system of the heart and a branching point of a left bundle branch (LBB) of the conduction system with the region, wherein the depth of the membranous septum comprises a depth of the cardiac conduction region.

26. The computer implemented method of claim 1, wherein computing the prediction comprises classifying the at least one parameter into a classification category selected from high likelihood of damage to the cardiac conduction system indicating a recommendation for pre-operative implantation of the pacemaker, and low likelihood of damage to the cardiac conduction system that does not justify the pre-operative implantation of the pacemaker.

27. A computer implemented method of training a machine learning model for predicting likelihood of a subject requiring a pacemaker after a TAVR procedure, comprising:
creating a training dataset of a plurality of records of a plurality of individuals, wherein a record of an individual includes:
at least one parameter computed from a sample 3D image of the individual, the at least one parameter selected from:
(i) a depth of a membranous septum computed as a distance between the membranous septum and a virtual annulus plane of a native aortic valve,
(ii) an angle of rotation of at least one cusp of the native aortic valve relative to the membranous septum,
(iii) a left ventricle (LV)—aorta angulation,
(iv) a depth of a conduction pathway computed as a distance between the conduction pathway overlying the membranous septum floor and a virtual annulus plane of a native aortic valve, wherein the conduction pathway is between a perforating bundle and a branching bundle, and
a ground truth indicating whether the individual required implantation of a pacemaker after the TAVR or did not require the pacemaker; and
training a machine learning model on the training dataset.

28. A system for predicting likelihood of a subject requiring a pacemaker after a transcatheter aortic valve replacement (TAVR) procedure, comprising:
at least one processor executing a code for:
computing from a 3D image of a subject at least one parameter selected from:
(i) a depth of a membranous septum computed as a distance between the membranous septum and a virtual annulus plane of a native aortic valve,
(ii) an angle of rotation of at least one cusp of the native aortic valve relative to the membranous septum,
(iii) a left ventricle (LV)—aorta angulation, and
(iv) a depth of a conduction pathway computed as a distance between the conduction pathway overlying the membranous septum floor and a virtual annulus plane of a native aortic valve, wherein the conduction pathway is between a perforating bundle and a branching bundle; and
computing a prediction of likelihood of the subject requiring the pacemaker after the TAVR according to the at least one parameter.
